(19) United States
(12) Patent Application Publication
(10) Pub. No.: US 2008/0146845 A1
(43) Pub. Date: Jun. 19, 2008

(54) Process for preparing Cinacalcet

(76) Inventors: Boaz Gome, Rishon-Lezion (IL); Leonid Levenfeld, Petach-Tikva (IL)

Correspondence Address: KENYON & KENYON LLP, ONE BROADWAY, NEW YORK, NY, 10004, US

(21) Appl. No.: 11/986,328

(22) Filed: Nov. 20, 2007

Related U.S. Application Data
(60) Provisional application No. 60/860,424, filed on Nov. 20, 2006.

(51) Int. Cl. C07C 211/01

(52) U.S. Cl. 564/337

(57) ABSTRACT

The invention provides a process for preparing Cinacalcet base in which a compound VI, having the structure:

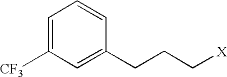

where X is C₁₋₃ alkylsulfonate, substituted and non-substituted C₆₋₁₀ aryl sulfonate or halogen, is reacted with (R)-1-Naphthylethylamine and, optionally, a base: 1) under minimal solvent conditions, 2) at a minimum temperature of about 100° C., preferably, greater than 121° C., and, more preferably, at a temperature of greater than 121° C. to about 130° C., and 3) under elevated pressure.

### RELATED APPLICATION

This application claims benefit of U.S. Provisional Patent Application No. 60/860,424, filed Nov. 20, 2006, the contents of which are incorporated herein in their entirety by reference.

### FIELD OF INVENTION

The invention is directed to a process for preparing Cinacalcet, (R)-α-methyl-N-[3-[3-(trifluoromethyl)phenyl]propyl]-1-naphthalenemethane amine.

### BACKGROUND OF THE INVENTION

(R)-α-methyl-N-[3-[3-(trifluoromethyl)phenyl]propyl]-1-naphthalenemethane amine (herein “Cinacalcet” or “CNC”) has a CAS number of 226256-56-0, a formula of C₂₂H₂₂F₃N, and is a free base, having the following structure:

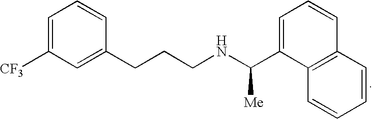

The hydrochloride salt of Cinacalcet is Cinacalcet hydrochloride (herein “Cinacalcet HCl” or “CNC-HCl”), having a CAS number of 364782-34-3, and the following structure:

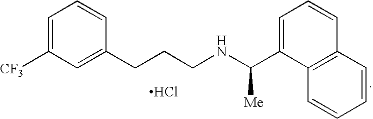

Cinacalcet HCl is marketed as SENSIPAR™, and is the first drug in a class of compounds known as calcimimetics to be approved by the FDA.

Calcimimetics are a class of orally active, small molecules that decrease the secretion of parathyroid hormone (PTH) by activating calcium receptors. The secretion of PTH is normally regulated by the calcium-sensing receptor. Calcimimetic agents increase the sensitivity of this receptor to calcium, which inhibits the release of parathyroid hormone, and lowers parathyroid hormone levels within a few hours. Calcimimetics are used to treat hyperparathyroidism, a condition characterized by the over-secretion of PTH that results when calcium receptors on parathyroid glands fail to respond properly to calcium in the bloodstream. Elevated levels of PTH are an indicator of secondary hyperparathyroidism associated with altered metabolism of calcium and phosphorus, bone pain, fractures, and an increased risk for cardiovascular death. As a calcimimetic, CNC-HCl is approved for treatment of secondary hyperparathyroidism in patients with chronic kidney disease on dialysis. Treatment with CNC-HCl lowers serum levels of PTH as well as the calcium/phosphorus ion product, a measure of the amount of calcium and phosphorus in the blood.

U.S. Pat. No. 6,011,068 discloses inorganic ion receptor activity, especially calcium receptor-active molecules, such as those having the general structure of Cinacalcet.

U.S. Pat. No. 6,211,244 discloses calcium receptor-active compounds related to Cinacalcet and methods of making such compounds. In accordance with the patent, Cinacalcet may be produced according to Scheme 1:

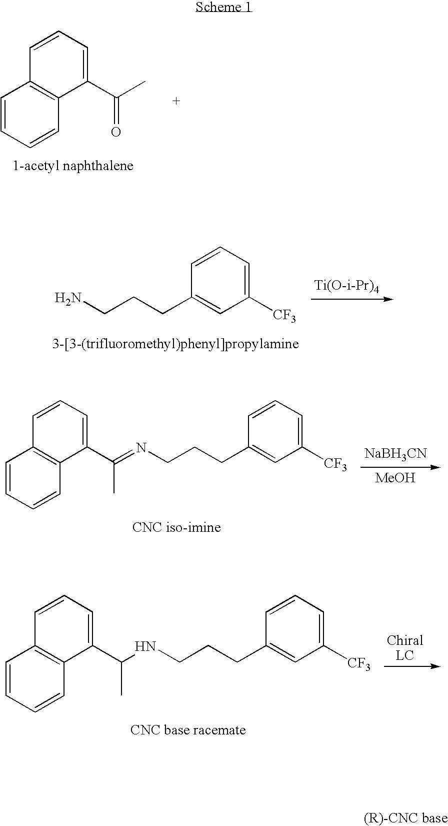

Similarly, using the process disclosed in U.S. Pat. No. 6,211,244, as well as DRUGS OF THE FUTURE (2002) 27 (9): 831, the desired Cinacalcet enantiomer may be produced according to scheme 2:

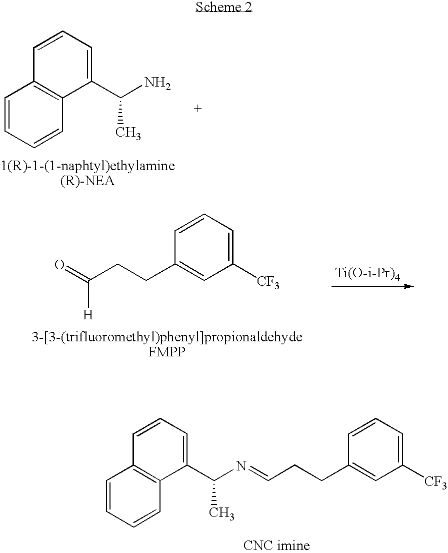

U.S. Pat. No. 6,211,244 discloses an additional process for the synthesis of Cinacalcet as described in Scheme 3:

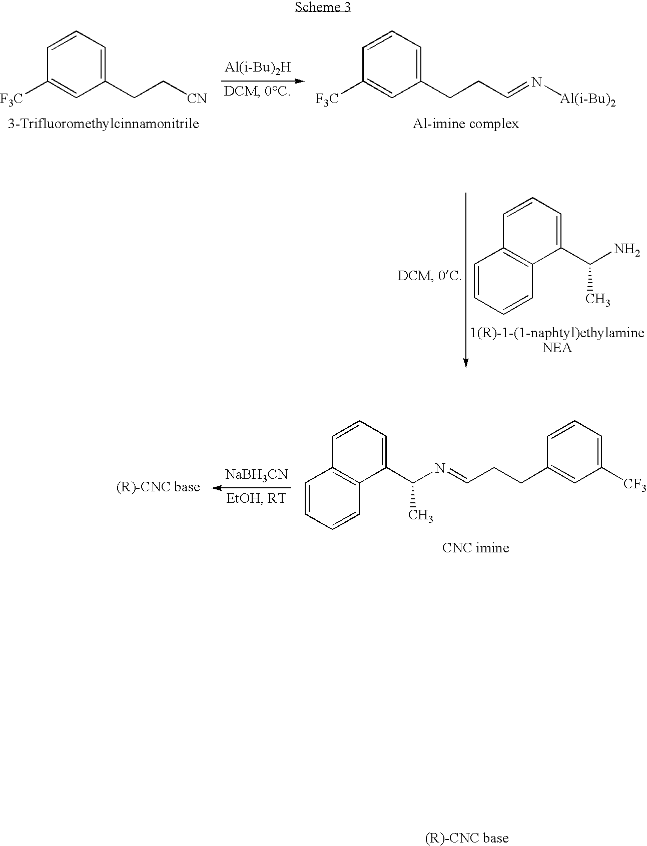

The above processes, require the use of reagents such as titanium isopropoxide which is highly hygroscopic and expensive, as well as toxic, and ethanolic or methanolic sodium cyanoborohydride, which is highly toxic and flammable, and not environmentally friendly, making the processes difficult to apply on industrial scale. In addition, the description of these processes is not detailed.

Moreover, the only synthetic route known for the precursor of the process described in Scheme 2, namely the 3-[3-(trifluoromethyl)phenyl]propionaldehyde (FMPP) is disclosed in footnote 12 of Tetrahedron Letters (2004) 45: 8355, and is described in Scheme 4:

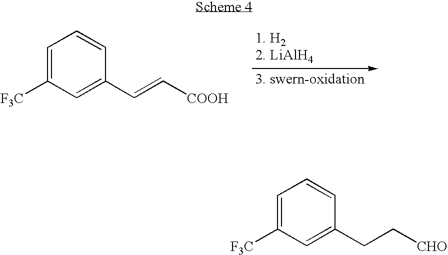

wherein reduction of the double bond of the corresponding cinnamic acid derivative, followed by reduction of the carboxylic acid moiety to the corresponding alcohol, which is then oxidized to the aldehyde by Swem-oxidation. The Swem-oxidation reaction involves the use of reagents, such as oxalyl chloride and DMSO, which are not environmentally friendly, and does not result in high yield, making the process arduous to apply on an industrial scale.

U.S. Pat. No. 7,250,533, discloses a process for preparing Cinacalcet, comprising: converting compound V of the structure:

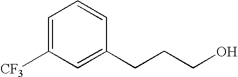

into compound VI of the structure:

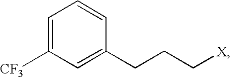

where X is a good leaving group, and converting compound VI to Cinacalcet base by preparing a solution of compound VI, (R)-1-Naphthylethylamine (R-NEA), and a base in an organic solvent, and maintaining the reaction mixture at a temperature of about 500 to about 120° C. for at least a sufficient period of obtain Cinacalcet base. However, improved processes for the preparation of cinacalcet base are constantly needed.

### SUMMARY OF THE INVENTION

The invention is directed to a process for preparing Cinacalcet base in which a compound VI, having the structure:

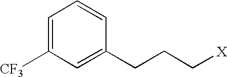

where X is C₁₋₃ alkylsulfonate, substituted and non-substituted C₆₋₁₀ aryl sulfonate or halogen, is reacted with (R)-1-Naphthylethylamine (R-NEA) and, optionally, a base: 1) under minimal solvent conditions, preferably, in the absence of any significant amount of solvent, 2) at a minimum temperature of about 100° C., preferably, greater than 121° C., and 3) under elevated pressure.

Preferably, the process further comprises a work-up procedure or step, which is presented for the preparation of Cinacalcet base containing less than 0.2 area percent R-NEA. The work-up procedure comprises:

(a) providing a solution of Cinacalcet base in a solvent which dissolve both Cinacalcet base and Cinacalcet HCl;

(b) washing the solution with water;

(c) separating the aqueous phase;

(d) acidifying solution to obtain a pH of about 0 to 2;

(e) neutralizing the organic phase to obtain a pH of about 7 to about 8.5; and;

(f) recovering the substantially free of R-NEA Cinacalcet base.

In another embodiment, the invention further provides a process for preparing Cinacalcet base comprising: combining compound VI, R-NEA, a base, and a solvent selected from the group consisting of toluene, xylene and chlorobenzene, at a reaction temperature of about 120° to about 130° C., and, preferably, at a temperature of greater than 121° C. to about 130° C.

In yet another embodiment, the invention provides a process for preparing Cinacalcet base comprising: combining compound VI, (R)-1-Naphthylethylamine (herein R-NEA), a base and a solvent selected from the group consisting of toluene/water and acetonitrile/water, under elevated pressure; preferably a pressure of about 3.5 bar to about 6 bar. Preferably the reaction temperature at elevated pressure is at least about 120° C., more preferably, from about 120° to about 150° C., and, most preferably, at a temperature of greater than 121° C. to about 140° C.

### DETAILED DESCRIPTION OF THE INVENTION

As used herein, the term “bar” refers to the gauge pressure for the pressure in bar, i.e., barg. As is known in the art, gauge pressure is the difference between the absolute pressure and the ambient atmospheric pressure. Therefore, the absolute pressure is equal to the gauge pressure plus the atmospheric pressure. That is, the absolute pressure, measured in bar, is substantially equivalent to the pressure in barg plus 1 bar. One bar is 1×10⁵ Pascals (Pa).

As used herein, compound VI refers to the following structure:

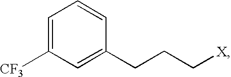

where X is C₁₋₃ alkyl sulfonate, substituted and non-substituted C₆₋₁₀ aryl sulfonate or halogen. In one preferable embodiment, compound VI is 3-(3-(trifluoromethyl)phenyl) propyl methanesulfonate (FTOMs).

As used herein, the term “R-NEA” refers to (R)-1-Naphthylethylamine.

In the processes of the present invention, when present, the base can be an organic or inorganic base. The organic or inorganic base can be selected from the group consisting of an amine or alkali carbonate. Preferably, the base is selected from the group consisting of K₂CO₃, NaHCO₃, Na₂CO₃, KHCO₃, and tertiary amines, such as triethylamine and diisopropylethyl amine. Most preferably, the base is K₂CO₃.

In the processes of the present invention, when a solvent is used, it may be selected from the group consisting of a C₆-C₈ aromatic hydrocarbon, C₁-C₄ alcohol, C₃-C₆ ester, C₃-C₆ ketone, acetonitrile, and mixtures of thereof with water. More preferably, the C₆-C₈ aromatic hydrocarbon is toluene. More preferably, the C₁-C₄ alcohol is selected from the group consisting of ethanol and isopropyl alcohol. More preferably, the C₃-C₆ ester is ethyl acetate. More preferably, the C₃-C₆ ketone is selected from the group consisting of methylisobutyl ketone (MIBK) and acetone. Most preferably, the organic solvent is toluene, acetonitrile, a mixture of water and toluene or a mixture of water and acetonitrile.

The present invention relates to new processes for preparing Cinacalcet base from compound VI, (R)-1-Naphthylethylamine (herein R-NEA) and, optionally, a base: 1) under minimal solvent conditions, 2) at a minimum temperature of about 100° C., preferably, greater than 121° C., and more preferably, at a temperature of greater than 121° C. to about 130° C., and 3) under elevated pressure. These processes are illustrated in scheme 5:

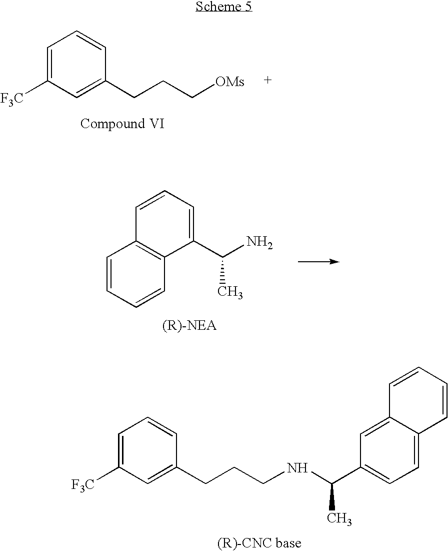

These processes improve the reaction kinetics compared to the prior art references. In addition, these reactions enable easier and faster work-up procedure. This improvement saves both the cost of the solvent, disposal of waste, and cost of performing evaporation. The work in these processes reduces the number of volumes used in the reaction mixture and as a result, the throughput of the production reactors increases.

In one embodiment, the invention provides a process for preparing Cinacalcet base comprising: combining compound VI with R-NEA and, optionally, a base under minimal solvent conditions, i.e., in less than about 2 ml of solvent per gram of compound VI. In one example, the amount of solvent is less than about 1.9 ml per gram of compound VI, preferably less than about 1.7 ml per gram of compound VI, more preferably less than about 1.5 ml per gram of compound VI, most preferably less than 1.0 ml per gram of compound VI. Excellent results have been obtained under neat conditions, i.e., in the substantial absence of solvent. The reaction temperature is typically about 85° C. to about 160° C. Preferably, the temperature is about 95° C. to about 140° C. More preferably, the reaction temperature is about 105° C. to about 130° C. When a solvent is used, preferable solvents include toluene, xylene, chlorobenzene, or mixtures thereof. The reaction is typically monitored so that more than 80 percent cinacalcet base is converted. Typically, this is after about 1 hour to about 10 hours.

The invention further provides a process for preparing Cinacalcet base comprising: combining compound VI, R-NEA, and, optionally, a base at a temperature of at least 100° C., preferably, greater than 121° C., and, more preferably, at a temperature of greater than 121° C. to about 130° C. Performing the reaction at this elevated temperature decreases reaction times by an amount significantly greater than would be expected. In one preferred embodiment, the reaction can be completed in as little as about one to three hours. When maximum yield is a significant consideration, the reaction time is preferably from about 5 hours to about 9 hours, more preferably, from about 5 hours to about 8 hours. If a solvent is used, useful solvents include toluene, xylene, and chlorobenzene.

The invention provides a process for preparing Cinacalcet base comprising: combining compound VI, (R)-1-Naphthylethylamine (R-NEA), and, optionally a base under elevated pressure. Elevated pressure may be from about 3.5 bars to about 10 bars, preferably about 3.5 to about 8 bars, more preferably about 3.5 to about 6 bars. Preferably, the reaction is performed at a temperature of at least about 120° C., more preferably, at a temperature of from about 120° to about 150° C., and, most preferably, at a temperature of from about 121° to about 140° C. Preferably, the pressure is of about above 3.5 bars to about 4.5 bars. Preferably, the reaction is for about 1 hour to about 10 hours, more preferably, for about 2 hours to about 5 hours.

Preferably, the processes described above further comprise a work-up step according to U.S. Pat. No. 7,250,533, the teachings of which are incorporated herein by reference in their entirety. The work-up procedure may comprise:

(a) providing a solution of Cinacalcet base in a solvent which dissolve both Cinacalcet base and Cinacalcet HCl;

(b) washing the solution with water;

(c) separating the aqueous phase;

(d) acidifying solution to obtain a pH of about 0 to 2;

(e) neutralizing the organic phase to obtain a pH of about 7 to about 8.5; and;

(f) recovering the substantially free of R-NEA Cinacalcet base.

Preferably, heating prior to step (d), to about 50° C. to about 80° C., is performed. Preferably, the solvent is selected from the group consisting of toluene, ethyl acetate, dichloromethane (DCM), chloroform, 1,2-dichloroethane, carbon tetrachloride, isobutyl acetate, xylene, benzene or mixtures thereof. More preferably, the solvent is selected from the group consisting of toluene, ethyl acetate, DCM, and mixtures thereof. Preferably the solvent is in a sufficient amount to obtain a solution. For example, when toluene is used, about 4 to about 7 volumes per gram of residue would be suitable. Preferably, the solution is acidified by the addition of an acid, such as hydrochloric acid. The pH is adjusted to about 7 to about 8.5, preferably, by washing with about 1.5 volumes of water about 2 or 3 times, and then with two volumes of a saturated solution of NaHCO₃ (1×2 volumes per gram of residue after evaporation). Preferably, the Cinacalcet base is recovered by washing with water (1×1.5 volumes per gram of residue after evaporation), and then evaporating the solvent under reduced pressure.

The conversion of CNC-base to CNC-HCl may be by any method known in the art. Preferably, the conversion of CNC-base to CNC-HCl is according to the U.S. Pat. No. 7,247,751, the teachings of which are incorporated herein by reference in their entirety.

### EXAMPLES

### Example 1A

### Preparation of CNC-Base Under Neat Conditions

A 1 liter glass lab reactor equipped with mechanical stirrer, and controlled heating/cooling system was purged with Nitrogen. The continuous flow of Nitrogen was maintained during the reaction step. 41.5 g of 3-(3-(trifluoromethyl)phenyl)propyl methanesulfonate (assay 96.4 percent; 40 g on 100 percent basis), 24.3 g of R-Naphthyl ethyl amine, and 9.8 g of K₂CO₃ were charged into the reactor. The stirrer was turned on, and the reactor jacket was heated to Tⱼ=105° C. The reaction mixture was stirred for 5 hours, and then sampled for end of reaction monitoring. The conversion to Cinacalcet-base: 85.3 percent

After 6 hours at 105° C., the reactor was cooled to 25° C., and 280 ml of toluene were charged. Then, the solution was filtered under reduced pressure, and the filter cake was washed with 80 ml of toluene. The reactor was washed with water, and the toluene solution was charged into the clean reactor. The reactor was heated to 70° C., and 100 ml of a 10 percent aqueous solution of hydrochloric acid was charged into the reactor. The mixture was stirred for 15 minutes, the mixer was stopped letting the phases separate, and the aqueous phase was then drained. The pH of the aqueous phase was tested, and found to be 0 to 1. The organic phase was washed with water two times. Each wash consisted of charging 120 ml of water, stirring for 15 minutes, stopping the mixer, letting the phases separate, and draining the aqueous phase. The pH of each aqueous phase was tested and found to be 0 to 1. After the aqueous washes, the organic phase was sampled and tested by HPLC. Then, 100 ml of a 10 percent solution of NaHCO₃ was charged into the reactor. The mixture was stirred for 30 minutes, the mixer was stopped letting the phases separate, and the aqueous phase was then drained. The pH of the aqueous phase was tested, and found to be 7 to 8. Then, 120 ml of water were charged into the reactor. The mixture was stirred for 15 minutes, the mixer was stopped letting the phases separate, and the aqueous phase was then drained. The pH of the aqueous phase was tested, and found to be 6 to 7. The organic phase in the reactor was cooled to 35° C., and the toluene was then evaporated under reduced pressure, at a maximum jacket temperature of 65° C. 44.5 g of Cinacalcet base were obtained.

### Example 1B

### Preparation of CNC HCl

A 20 g sample of the Cinacalcet-base obtained in example 1A was charged into a 1 liter glass lab reactor equipped with mechanical stirrer, and controlled heating/cooling system. 300 ml (15 volumes vs. CNC-base) of methyl t-butyl ether (MTBE) were charged, the mixer was turned on, and a solution was obtained. The temperature was adjusted to 25° C., and hydrochloric acid gas was introduced into the reactor during 20 minutes, until a pH of 1 to 2 was measured. During the introduction, the reactor temperature has increased to a maximum of 29° C. The obtained CNC-HCl precipitated, providing a slurry. The slurry was stirred for 1 hour, and the product was then isolated by filtration under reduced pressure. The reactor was washed with 20 ml of MTBE, and the wash liquor was used to wash the filter cake. Then, the filter cake was washed with 40 ml of MTBE. The wet product was dried in a tray oven at 50° C., under reduced pressure. 15.6 g of dry Cinacalcet-HCl were obtained.

### Example 2A

### Preparation of CNC-Base Under Neat Conditions without Using a Base

1 liter glass lab reactor equipped with mechanical stirrer, and controlled heating/cooling system was purged with Nitrogen. The continuous flow of Nitrogen was maintained during the reaction step.

43.2 g of 3-(3-(trifluoromethyl)phenyl)propyl methanesulfonate (assay 92.6 percent; 40 g on 100 percent basis) and 24.3 g of R-Naphthyl ethyl amine were charged into the reactor. The stirrer was turned on, and the reactor jacket was heated to Tⱼ=105° C. The reaction mixture was stirred for 5 hours, and the reaction mixture was then sampled for end of reaction monitoring. The conversion to Cinacalcet-base: 77.5 percent. After 7 hours at 105° C., the reaction mixture was sampled again, and tested for end of reaction monitoring. The conversion to Cinacalcet-base: 80.3 percent. After 8.5 hours at 105° C. the solution was cooled, and 280 ml of toluene were charged. Then, the reactor temperature was maintained at 70° C., and 100 ml of a 10 percent aqueous solution of hydrochloric acid was charged into the reactor. The mixture was stirred for 15 minutes, the mixer was stopped letting the phases separate, and the aqueous phase was then drained. The pH of the aqueous phase was tested, and found to be 0. The organic phase was washed with water two times. Each wash consisted of charging 120 ml of water, stirring for 15 minutes, stopping the mixer, letting the phases separate, and draining the aqueous phase. The pH of the aqueous phases was tested and found to be 1, and 1 to 2 respectively. After the aqueous washes, the organic phase was sampled and tested by HPLC. Then, 100 ml of a 10 percent solution of NaHCO₃ was charged into the reactor. The mixture was stirred for 30 minutes, the mixer was stopped letting the phases separate, and the aqueous phase was then drained. The pH of the aqueous phase was tested, and found to be 9. Then, 120 ml of water were charged into the reactor. The mixture was stirred for 15 minutes, the mixer was stopped letting the phases separate, and the aqueous phase was then drained. The pH of the aqueous phase was tested, and found to be 7. The organic phase in the reactor was cooled, and the toluene was then evaporated under reduced pressure, at a maximum jacket temperature of 65° C.

47.7 g of Cinacalcet base were obtained.

### Example 2B

### Preparation of Cinacalcet HCl

20 g of Cinacalcet-base obtained in example 2A were charged into a 1 liter glass lab reactor equipped with mechanical stirrer, and controlled heating/cooling system. 300 ml (15 volumes vs. Cinacalcet-base) of methyl t-butyl ether (MTBE) were charged, the mixer was turned on, and a solution was obtained. The temperature was adjusted to 25° C., and hydrochloric acid gas was introduced into the reactor during 25 minutes, until a pH of 1 to 2 was measured. During the introduction, the reactor temperature has increased to a maximum of 30° C. The obtained Cinacalcet-HCl precipitated, providing a slurry. The slurry was stirred for 1 hour, and the product was then isolated by filtration under reduced pressure. The reactor was washed with 20 ml of MTBE, and the wash liquor was used to wash the filter cake. Then, the filter cake was washed with 40 ml of MTBE. The wet product was dried in a tray oven at 50° C., under reduced pressure. 14.3 g of dry Cinacalcet-HCl were obtained.

### Example 3A

### Preparation of CNC-Base Using Toluene

1 liter glass lab reactor equipped with mechanical stirrer, and controlled heating/cooling system was purged with Nitrogen. The continuous flow of Nitrogen was maintained during the reaction step.

42.1 g of 3-(3-(trifluoromethyl)phenyl)propyl methanesulfonate (FTOMs) (assay 94.7 percent; 40 g on 100 percent basis), 24.3 g of R-Naphthyl ethyl amine, 9.8 g K₂CO₃, and 40 ml toluene (1 volume vs. FTOMs) were charged into the reactor. The stirrer was turned on, and the reactor jacket was heated to Tⱼ=130° C. The obtained reflux temperature: 118 to 122° C. The reaction mixture was stirred for 3 hours, and then sampled for end of reaction monitoring. The conversion to Cinacalcet-base: 85.3 percent. The reaction continued for an additional 2.5 hours (total 5.5 hours), and, then, the reaction mixture was cooled to 25° C., and 240 ml of toluene (6 volumes) and 40 ml water (1 volume) were charged. The two phases were stirred, and the aqueous phase was then separated. The organic phase was sampled, and it was found that the conversion to CNC-base was: 91.0 percent.

The organic phase was heated to 70° C., and, and 80 ml of a 10 percent aqueous solution of hydrochloric acid was charged into the reactor. The mixture was stirred for 15 minutes, the mixer was stopped letting the phases separate, and the aqueous phase was then drained. The pH of the aqueous phase was tested, and found to be 0 to 1. The organic phase was washed with water two times. Each wash consisted of charging 120 ml of water, stirring for 15 minutes, stopping the mixer, letting the phases separate, and draining the aqueous phase. The pH of the aqueous phases was tested and found to be 1 to 2 and 1 to 2 respectively. Then, 80 ml of a 10 percent solution of NaHCO₃ was charged into the reactor. The mixture was stirred for 15 minutes, the mixer was stopped letting the phases separate, and the aqueous phase was then drained. The pH of the aqueous phase was tested, and found to be 7 to 8. Then, 120 ml of water were charged into the reactor. The mixture was stirred for 15 minutes, the mixer was stopped letting the phases separate, and the aqueous phase was then drained. The pH of the aqueous phase was tested, and found to be 7. The organic phase in the reactor was cooled to 15° C., and the toluene was then evaporated under reduced pressure, at a maximum jacket temperature of 65° C. 46.9 g of Cinacalcet base were obtained.

### Example 3B

### Preparation of Cinacalcet HCl

25 g of Cinacalcet-base obtained in example 3A were charged into a 1 liter glass lab reactor equipped with mechanical stirrer, and controlled heating/cooling system. 375 ml (15 volumes vs. Cinacalcet-base) of methyl t-butyl ether (MTBE) were charged, the mixer was turned on, and a solution was obtained. The temperature was adjusted to 25° C., and hydrochloric acid gas was introduced into the reactor, until a pH of 1 to 2 was measured. The obtained Cinacalcet-HCl precipitated, providing a slurry. The slurry was stirred for 1 hour, and the product was then isolated by filtration under reduced pressure. Then, the filter cake was washed with MTBE three times, 20 ml of MTBE each wash. The wet product was dried in a tray oven at 50° C., under reduced pressure. 22.5 g of dry Cinacalcet-HCl were obtained.

### Example 4A

### Preparation of Cinacalcet-Base Starting at Elevated Pressure in Acetonitrile/Water

A 2 liter stainless steel lab reactor, equipped with mechanical stirrer and controlled heating/cooling system, was purged with Nitrogen.

84.2 g of 3-(3-(trifluoromethyl)phenyl)propyl methanesulfonate (Assay=95.0, 80 g on 100 percent basis), 19.6 g K₂CO₃, 48.6 g of R-Naphthyl ethyl amine 320 ml of Acetonitrile, and 96 ml of water were charged into the reactor. The stirrer was turned on, the vent valve was closed, and the reactor jacket was heated to Tⱼ=140° C. The reactor pressure was increased up to 6.2 bar, and the reactor temperature has increased to 131.6° C. After 2 hours the reaction mixture was sampled for end of reaction monitoring. It was found that the conversion to Cinacalcet was 86 percent. After an additional 3 hours (total 5 hours), the reaction mixture was cooled to 25° C., and the excess pressure (3.4 bar, due to CO₂ emission) was released. The aqueous phase was separated, and the organic phase was transferred into a 1 liter glass lab reactor. The solvent was evaporated under reduced pressure, at a maximum jacket temperature of 40° C. The reactor was cooled to 25° C., and 560 ml of toluene were charged.

Then, the reactor temperature was maintained at 70° C., and 160 ml of a 10 percent aqueous solution of hydrochloric acid were charged into the reactor. The mixture was stirred for 15 minutes, the mixer was stopped, letting the phases separate, and, then, the aqueous phase was drained. The pH of the aqueous phase was tested, and found to be 0 to 1. The organic phase was washed with water two times. Each wash consisted of charging 250 ml of water, stirring for 15 minutes, stopping the mixer, letting the phases separate, and draining the aqueous phase. The pH of the aqueous phases was tested and found to be 1 to 2. Then 160 ml of a 10 percent solution of NaHCO₃ were charged into the reactor. The mixture was stirred for 15 minutes, the mixer was stopped, letting the phases separate, and, then, the aqueous phase was drained. The pH of the aqueous phase was tested, and found to be 7 to 8. Then, 210 ml of water were charged into the reactor. The mixture was stirred for 15 minutes, the mixer was stopped, letting the phases separate, and then the aqueous phase was drained. The pH of the aqueous phase was tested, and found to be 6 to 7. The organic phase in the reactor was cooled, and, then, the toluene was evaporated under reduced pressure, at a maximum jacket temperature of 65° C. 90 g of Cinacalcet base were obtained.

### Example 4B

### Preparation of Cinacalcet HCl

25 g of Cinacalcet-base obtained in experiment 4A were charged into a 1 liter glass lab reactor equipped with mechanical stirrer, and controlled heating/cooling system. 375 ml (15 volumes vs. Cinacalcet-base) of methyl t-butyl ether (MTBE) were charged, the mixer was turned on, and a solution was obtained. The temperature was adjusted to 25° C., and hydrochloric acid gas was introduced into the reactor over a period of 25 minutes, until a pH of 1 to 2 was measured. During the introduction, the reactor temperature has increased to a maximum of 30° C. The obtained Cinacalcet-HCl precipitated, providing a slurry. The slurry was stirred for 1 hour, and, then, the product was isolated by filtration under reduced pressure. The filter cake was washed with 50 ml MTBE. The wet product was dried in a tray oven at 50° C., under reduced pressure. 20.8 g of dry Cinacalcet-HCl were obtained.

### Example 5A

### Preparation of Cinacalcet-Base at Elevated Pressure in Toluene/Water

A 2 liter stainless steel lab reactor, equipped with mechanical stirrer, and controlled heating/cooling system, was purged with Nitrogen.

84.2 g of 3-(3-(trifluoromethyl)phenyl)propyl methanesulfonate (Assay=95.0, 80 g on 100 percent basis), 19.6 g K₂CO₃, 48.6 g of R-Naphthyl ethyl amine, 160 ml of toluene, and 96 ml of water were charged into the reactor. The stirrer was turned on, the vent valve was closed, and the reactor jacket was heated to Tⱼ=140° C. The reactor pressure has increased up to 7.2 bar, and the reactor temperature has increased to 133° C. After 2 hours, the reaction mixture was sampled for end of reaction monitoring. It was found that the conversion to Cinacalcet-base was 85.2 percent. After an additional 2 hours (total 4 hours), the reaction mixture was sampled again, and it was found that the conversion to Cinacalcet-base was 89.5 percent.

Then reaction mixture was cooled to 25° C., and the excess pressure was released. The aqueous phase was separated, 400 ml of toluene were charged, and the organic phase was transferred into a 1 liter glass lab reactor.

Then, the reactor temperature was maintained at 70° C., and 160 ml of a 10 percent aqueous solution of hydrochloric acid was charged into the reactor. The mixture was stirred for 15 minutes, the mixer was stopped letting the phases separate, and then the aqueous phase was drained. The pH of the aqueous phase was tested, and found to be 0 to 1. The organic phase was washed with water two times. Each wash consisted of charging 250 ml of water, stirring for 15 minutes, stopping the mixer, letting the phases separate, and draining the aqueous phase. The pH of the aqueous phases was tested and found to be 1 to 2. Then, 160 ml of a 10 percent solution of NaHCO₃ was charged into the reactor. The mixture was stirred for 15 minutes, the mixer was stopped letting the phases separate, and then the aqueous phase was drained. The pH of the aqueous phase was tested, and found to be 7 to 8. Then, 250 ml of water were charged into the reactor. The mixture was stirred for 15 minutes, the mixer was stopped letting the phases separate, and then the aqueous phase was drained. The pH of the aqueous phase was tested, and found to be 6 to 7. The organic phase in the reactor was cooled, and then the toluene was evaporated under reduced pressure, at a maximum jacket temperature of 65° C. 79.9 g of Cinacalcet base were obtained.

### Example 5B

### Preparation of Cinacalcet HCl

25 g of Cinacalcet-base obtained in example 5A were charged into a 1 liter glass lab reactor equipped with mechanical stirrer, and controlled heating/cooling system. 375 ml (15 volumes vs. Cinacalcet-base) of methyl t-butyl ether (MTBE) were charged, the mixer was turned on, and a solution was obtained. The temperature was adjusted to 25° C., and hydrochloric acid gas was introduced into the reactor over a period of 25 minutes, until a pH of 1 to 2 was measured. During the introduction, the reactor temperature has increased to a maximum of 30° C. The obtained Cinacalcet-HCl precipitated, providing a slurry. The slurry was stirred for 1 hour, and then the product was isolated by filtration under reduced pressure. The reactor was washed with 25 ml of MTBE, and the wash liquor was used to wash the filter cake. The filter cake was washed with 50 ml MTBE. The wet product was dried in a tray oven at 50° C., under reduced pressure. 21.5 g of dry Cinacalcet-HCl were obtained

### Example 6A

### Preparation of Cinacalcet-Base Starting with FTOMs Solution, and Evaporating During the Reaction

231 Kg of FTOMs solution in toluene (41.2 Kg on 100 percent basis), 10.2 Kg K₂CO₃, and 24.9 Kg of R-Naphthyl ethyl amine were charged in to a stainless steel reactor. The stirrer was turned on, and the reactor jacket was heated to Tⱼ=129.2° C.

The solvent was evaporated at atmospheric pressure, until the reactor boiling temperature has increased from 111.6° C. to 123.1° C. At these conditions, the volume of solvent remained in the reactor is about 1 volume.

The reaction mixture was stirred for 5 hours. During the reaction, the reactor temperature was 122-124° C. Then, the reactor was cooled 74.7° C., and 206.0 liter of toluene (˜5 vol), and 82 liter of water (˜2 vol), were charged. The two phases were stirred, and, then, the aqueous phase was separated. The organic phase was transferred into a glass lined reactor. The reactor temperature was maintained at 70° C., and 75 kg of water and 32.5 kg of a 32 percent hydrochloric acid were charged in to the reactor. The mixture was stirred, and, then, the mixer was stopped, letting the phases separate. The aqueous phase was drained. The pH of the aqueous phase was tested, and found to be 0. The organic phase was washed with water two times. Each wash consisted of charging 125 liter of water, stirring, stopping the mixer, letting the phases separate, and draining the aqueous phase. The pH of the aqueous phases was tested and found to be 1.6, and 1.2 respectively. The reactor was cooled top 23° C. Then, 125 liter of water, and, then, 15.2 kg of NaHCO₃ were charged in to the reactor. The mixture was stirred, the mixer was stopped, letting the phases separate, and then the aqueous phase was drained. The pH of the aqueous phase was tested, and found to be 8. Then, 125 liters of water were charged in to the reactor. The mixture was stirred, the mixer was stopped, letting the phases separate, and then the aqueous phase was drained. The pH of the aqueous phase was tested, and found to be 6.7. The organic phase was used for preparation of a batch of Cinacalcet-HCl as presented in example 6B.

### Example 6B

### Preparation of Cinacalcet HCl

344.9 Kg (48.6 kg on 100 percent basis) of Cinacalcet-base solution in toluene obtained in example 6A were charged in to a glass lined reactor equipped with mechanical stirrer, and controlled heating/cooling system. The solution was filtered through polishing filters into a second glass lined reactor. Then filtration system was washed with 84.6 liter of toluene. The solvent was evaporated at a pressure of 49 mm Hg and a maximum reactor temperature of 64.9° C. (Tⱼ=73.7° C.). After completion of the evaporation, the reactor was cooled to 25.2° C., and the pressure has increased to atmospheric. Then, 546.8 kg (15 volumes vs. Cinacalcet-base) of methyl t-butyl ether (MTBE) were charged, the mixer was turned on, and a solution was obtained. The temperature was adjusted to 25° C., and hydrochloric acid gas was introduced in to the reactor until a pH of 1.3 was measured. During the introduction, the reactor temperature was increased to a maximum of 21.2° C. The obtained Cinacalcet-HCl precipitated, so slurry was obtained. The slurry was stirred for about 1 hour, and, then, the product was isolated by filtration in a centrifuge.

The filter cake was washed with MTBE three times, 36.5 Kg of MTBE each wash. The wet product was dried in a stirred vacuum drier at 56° C., under reduced pressure. 41 Kg of dry Cinacalcet-HCl were obtained.

### Example 7A

### Preparation of Cinacalcet-Base Starting with 3-(3-(trifluoromethyl)phenyl)propyl Methanesulfonate Solution, and Evaporating During the Reaction

1 liter glass lab reactor, equipped with mechanical stirrer, and controlled heating/cooling system, was purged with Nitrogen.

366 g of 3-(3-(trifluoromethyl)phenyl)propyl methanesulfonate solution in toluene (68.5 g on 100 percent basis), 15.4 g K₂CO₃, and 33.3 g of R-Naphthyl ethyl amine were charged into the reactor. The stirrer was turned on, and the reactor jacket was heated to Tⱼ=140° C.

The solvent was evaporated at atmospheric pressure, until the reactor boiling temperature increased from 109° C. to 124° C. At these conditions, the volume of solvent remained in the reactor was about 1 volume.

The reaction mixture was stirred for 5 hours. During the reaction the reactor temperature was 121 to 123° C. Then, the reactor was cooled, and 420 ml of toluene (˜6 volumes) and 140 ml water (˜2 volumes) were charged. The two phases were stirred, and the aqueous phase was then separated.

Then, the reactor temperature was maintained at 70° C., and 171 ml of a 10 percent aqueous solution of hydrochloric acid were charged into the reactor. The mixture was stirred for 15 minutes, the mixer was stopped letting the phases separate, and the aqueous phase was then drained. The pH of the aqueous phase was tested, and found to be 0. The organic phase was washed with water two times. Each wash consisted of charging 210 ml of water, stirring for 15 minutes, stopping the mixer, letting the phases separate, and draining the aqueous phase. The pH of the aqueous phases was tested and found to be 1 and 1.6, respectively. Then, 200 ml of a 10 percent solution of NaHCO₃ were charged into the reactor. The mixture was stirred for 30 minutes, the mixer was stopped, letting the phases separate, and the aqueous phase was then drained. The pH of the aqueous phase was tested, and found to be 8.5. Then, 210 ml of water were charged into the reactor. The mixture was stirred for 15 minutes, the mixer was stopped letting the phases separate, and the aqueous phase was then drained. The pH of the aqueous phase was tested, and found to be 6.5 to 7. The organic phase in the reactor was cooled, and the toluene was then evaporated under reduced pressure, at a maximum jacket temperature of 65° C.

82 g of Cinacalcet base were obtained.

### Example 7B

### Preparation of Cinacalcet HCl

30 g of Cinacalcet-base obtained in example 7A were charged into a 1 liter glass lab reactor equipped with mechanical stirrer and controlled heating/cooling system. 450 ml (15 volumes vs. Cinacalcet-base) of methyl t-butyl ether (MTBE) were charged, the mixer was turned on, and a solution was obtained. The temperature was adjusted to 25° C., and hydrochloric acid gas was introduced into the reactor during 25 minutes, until a pH of 1.3 to 1.4 was measured. During the introduction, the reactor temperature has increased to a maximum of 30° C. The obtained Cinacalcet-HCl precipitated, providing a slurry. The slurry was stirred for 1 hour, and then the product was isolated by filtration under reduced pressure.

The filter cake was washed with MTBE three times, 30 ml of MTBE each wash. The wet product was dried in a tray oven at 50° C., under reduced pressure.

21.1 g of dry Cinacalcet-HCl were obtained.

What is claimed:

1. A process for the synthesis of Cinacalcet base, comprising:
combining a compound VI of formula 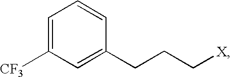
wherein X is a C₁₋₃ alkyl sulfonate, a substituted and non-substituted C₆₋₁₀ aryl sulfonate, or a halogen,
with (R)-1-Naphthylethylamine and, optionally, a base, in the presence of no more than about 2 ml of solvent per gram of compound VI, thereby forming Cinacalcet base.

2. The process of claim 1, wherein the solvent is present in an amount of no more than about 1.7 ml of solvent per gram of compound VI.

3. The process of claim 1, wherein the solvent is present in an amount of no more than about 1.5 ml of solvent per gram of compound VI.

4. The process of claim 1, wherein the solvent is present in an amount of no more than about 1 ml of solvent per gram of compound VI.

5. The process according to claim 1, wherein the compound of formula VI is reacted with (R)-1-Naphthylethylamine in the absence of any significant amount of a solvent.

6. The process according to claim 1, wherein the Cinacalcet base is formed at a reaction temperature of from about 85° to about 160° C.

7. The process according to claim 6, wherein the reaction temperature is from about 95° to about 140° C.

8. The process according to claim 6, wherein the reaction temperature is from about 105° to about 130° C.

9. The process according to claim 1, wherein the compound VI is 3-(3-(Trifluoromethyl)phenyl)propyl methanesulfonate.

10. The process according to claim 1, wherein the base is present, and is an amine or alkali carbonate.

11. The process according to claim 10, wherein the base is selected from the group consisting of tertiary amines, K₂CO₃, NaHCO₃, Na₂CO₃, and KHCO₃.

12. The process according to claim 10, wherein the base is triethylamine, diisopropylethyl amine, or K₂CO₃.

13. The process according to claim 10, wherein the base is K₂CO₃.

14. The process according to claim 1, further comprising forming the Cinacalcet base under a pressure of from about 3.5 bars to about 6 bars.

15. The process according to claim 1, further comprising removing residual (R)-1-Naphthylethylamine in a process comprising:
(a) providing a solution of the Cinacalcet base in a solvent that dissolves both Cinacalcet base and Cinacalcet HCl;
(b) washing the solution with water;
(c) separating the aqueous phase;
(d) acidifying solution to obtain a pH of about 0 to 2;
(e) neutralizing the organic phase to obtain a pH of about 7 to about 8.5; and;
(f) recovering Cinacalcet base having a (R)-1-Naphthylethylamine content of less than 0.2 area percent.

16. The process according to claim 15, further comprising heating to a temperature of from about 50° to about 80° C. prior to acidifying.

17. The process according to claim 15, wherein the solvent of the solution of the Cinacalcet base is selected from the group consisting of toluene, ethyl acetate, dichloromethane, chloroform, 1,2-dichloroethane, carbon tetrachloride, isobutyl acetate, xylene, benzene, and mixtures thereof.

18. The process according to claim 15, wherein the solvent of the solution of the Cinacalcet base is selected from the group consisting of toluene, ethyl acetate, dichlotomethane, and mixtures thereof.

19. A process for the synthesis of Cinacalcet base, comprising:
combining a compound VI of formula 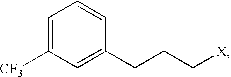
wherein X is a C₁₋₃ alkyl sulfonate, a substituted and non-substituted C₆₋₁₀ aryl sulfonate, or a halogen,
with (R)-1-Naphthylethylamine and, optionally, a base at a temperature greater than about 100° C., thereby forming Cinacalcet base.

20. The process according to claim 19, wherein the reaction temperature is greater than 121° C.

21. The process according to claim 19, wherein the reaction temperature is greater than 121° C. to about 130° C.

22. The process according to claim 19, wherein the compound VI is 3-(3-(Trifluoromethyl)phenyl)propyl methanesulfonate.

23. The process according to claim 19, wherein the base is present, and is an amine or alkali carbonate.

24. The process according to claim 23, wherein the base is selected from the group consisting of tertiary amines, K₂CO₃, NaHCO₃, Na₂CO₃, and KHCO₃.

25. The process according to claim 23, wherein the base is triethylamine, diisopropylethyl amine, or K₂CO₃.

26. The process according to claim 23, wherein the base is K₂CO₃.

27. The process according to claim 19, further comprising forming the Cinacalcet base under a pressure of from about 3.5 bars to about 6 bars.

28. The process according to claim 19, further comprising removing residual (R)-1-Naphthylethylamine in a process comprising:
(a) providing a solution of the Cinacalcet base in a solvent that dissolves both Cinacalcet base and Cinacalcet HCl;
(b) washing the solution with water;
(c) separating the aqueous phase;
(d) acidifying solution to obtain a pH of about 0 to 2;
(e) neutralizing the organic phase to obtain a pH of about 7 to about 8.5; and;
(f) recovering Cinacalcet base having a (R)-1-Naphthylethylamine content of less than 0.2 area percent.

29. A process for the synthesis of Cinacalcet base, comprising:
combining a compound VI of formula 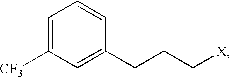
wherein X is a C₁₋₃ alkyl sulfonate, a substituted and non-substituted C₆₋₁₀ aryl sulfonate, or a halogen,
with (R)-1-Naphthylethylamine and, optionally, a base, under a pressure of from about 3.5 bars to about 6 bars, thereby forming Cinacalcet base.

30. The process according to claim 29, wherein the Cinacalcet base is formed at a reaction temperature of at least about 120° C.

31. The process according to claim 29, wherein the reaction temperature is from about 120° to about 150° C.

32. The process according to claim 29, wherein the reaction temperature is from at least 121° to about 140° C.

33. The process according to claim 29, wherein the compound VI is 3-(3-(Trifluoromethyl)phenyl)propyl methanesulfonate.

34. The process according to claim 29, wherein the base is present, and is an amine or alkali carbonate.

35. The process according to claim 34, wherein the base is selected from the group consisting of tertiary amines, K₂CO₃, NaHCO₃, Na₂CO₃, and KHCO₃.

36. The process according to claim 35, wherein the base is triethylamine, diisopropylethyl amine, or K₂CO₃.

37. The process according to claim 35, wherein the base is K₂CO₃.

38. The process according to claim 29, further comprising adding an organic solvent.

39. The process according to claim 38, wherein the organic solvent is selected from the group consisting of a C₆-C₈ aromatic hydrocarbon, a C₁-C₄ alcohol, a C₃-C₆ ester, a C₃-C₆ ketone, acetonitrile, and mixtures thereof with water.

40. The process according to claim 39, wherein the C₆-C₈ aromatic hydrocarbon is toluene.

41. The process according to claim 39, wherein the C₁-C₄ alcohol is selected from the group consisting of ethanol and isopropyl alcohol.

42. The process according to claim 39, wherein the C₃-C₆ ester is ethyl acetate.

43. The process according to claim 39, wherein the C₃-C₆ ketone is selected from the group consisting of methylisobutyl ketone and acetone.

44. The process according to claim 39, wherein the organic solvent is a mixture of water and toluene or a mixture of water and acetonitrile.

45. The process according to claim 29, wherein the pressure is above about 3.5 bars to about 4.5 bars.

46. The process according to claim 29, further comprising removing residual (R)-1-Naphthylethylamine in a process comprising:
(a) providing a solution of the Cinacalcet base in a solvent that dissolves both Cinacalcet base and Cinacalcet HCl;
(b) washing the solution with water;
(c) separating the aqueous phase;
(d) acidifying solution to obtain a pH of about 0 to 2;
(e) neutralizing the organic phase to obtain a pH of about 7 to about 8.5; and;
(f) recovering Cinacalcet base having a (R)-1-Naphthylethylamine content of less than 0.2 area percent.
